(19) United States
(12) Patent Application Publication
(10) Pub. No.: US 2025/0364110 A1
(43) Pub. Date: Nov. 27, 2025

(54) Utensil Using Artificial Intelligence For Meal Tracking

(71) Applicant: Mrida Enterprise, Inc., Allen, TX (US)

(72) Inventors: Vishal Modi, Allen, TX (US); Benjamin Yeffeth, Wylie, TX (US)

(73) Assignee: Mrida Enterprise, Inc., Allen, TX (US)

(21) Appl. No.: 19/212,969

(22) Filed: May 20, 2025

Related U.S. Application Data
(60) Provisional application No. 63/649,946, filed on May 21, 2024.

(51) Int. Cl. G16H 20/60; A47G 21/02; G06V 20/68

(52) U.S. Cl. CPC G16H 20/60; A47G 21/02; G06V 20/68

(57) ABSTRACT

A utensil for monitoring the eating habits of a user. The utensil comprises a handle; a head coupled to the handle that receives food; a camera module disposed in the handle that captures an image of food; and a load cell coupled to the head that measures force caused by food on the head. The utensil also includes an inertial measurement unit disposed in the handle that detects movement of the utensil and a control processor disposed in the handle. The control processor is configured to determine a weight of the food on the head based on force measurements received from the load cell.

### CROSS-REFERENCE TO RELATED APPLICATION(S) AND CLAIM OF PRIORITY

The present application is related to U.S. Provisional Patent No. 63/649,946, filed 21 May 2024, entitled “Utensil Using AI For Meal Tracking”. Provisional Patent No. 63/649,946 is assigned to the assignee of the present application and is hereby incorporated by reference into the present application as if fully set forth herein. The present application hereby claims priority under 35 U.S.C. § 119(e) to U.S. Provisional Patent No. 63/649,946.

### TECHNICAL FIELD

The present application relates generally to eating utensils and, more specifically, to an apparatus and method using artificial intelligence to track meals.

### BACKGROUND

Computer-aided technology has been used for meal and nutrition tracking. These technologies include web-based food-logging applications, mobile applications, and even SMS-based solutions. Technical improvements have reduced some of the problems of paper-based food tracking, but the process is still filled with inaccuracies and inefficiencies. Some innovations have implemented AI-based image recognition techniques that help identify the type of food being consumed. However, the onus remains on the user to estimate the portion of food being consumed. According to a study conducted by NIH, consumers may underestimate their caloric intake by up to 30%. Additionally, many Americans eat food at a much faster pace than medically recommended. One study found that 42% of children whose parents reported that the children ate quickly were overweight and these children were also more likely to show overeating behaviors. Existing meal-tracking solutions do not track eating speed.

Therefore, there is a need for improved systems and methods for tracking the eating habits of people. In particular, there is a need for systems and methods that accurately record the food consumption of users.

### SUMMARY

To address the above-discussed deficiencies of the prior art, it is a primary object of the present disclosure to provide a method of determining bite events based on capacitive detection of mouth contact using a capacitive sense tuning system for removable heads using dynamically adjusted capacitance. The disclosed system and method calculates bite weight via pre-bite and post-bite changes (i.e., delta values) with real-time smoothing.

The disclosed system and method compensates weight readings for forces imposed by user movement. The system transmits food images from a camera mounted on a utensil to a mobile application for classification. It is a primary object of the present disclosure to provide a multi-MCU sensor integrated system for real-time eating behavior tracking, including use of bite detection to track intervals between user bites Dand further including use of haptic feedback (in a utensil) as a mechanism for real-time nutritional intervention.

To address the above-discussed deficiencies of the prior art, it is a primary object of the present disclosure to provide a utensil for monitoring the eating habits of a user. The utensil comprises: i) a handle; ii) a head coupled to the handle and configured to receive food; iii) a camera module disposed in the handle and configured to capture an image of food on a plate; and iv) a load cell coupled to the head and configured to measure force caused by food on the head. The utensil further includes: v) an inertial measurement unit disposed in the handle and configured to detect movement of the utensil; and vi) a control processor disposed in the handle, wherein the control processor is configured to determine a weight of the food on the head based on force measurements received from the load cell.

In an embodiment, the head includes a spoon head configured to be removably coupled to the handle.

In another embodiment, the head includes a fork head configured to be removably coupled to the handle.

In yet another embodiment, the head further comprises a capacitive touch sensor configured to detect contact between the head and the mouth of a user.

In still another embodiment, the handle further comprises a radio transceiver.

In a further embodiment, the control processor is further configured to receive a captured image of food from the camera and to process the captured image using an image recognition algorithm in the handle.

In a yet further embodiment, the control processor is further configured to receive a captured image of food from the camera and to transmit the captured image to an image recognition server via the radio transceiver.

In a still further embodiment, the control processor is further configured to determine a time period between user bites based on the force measurements received from the load cell.

In an embodiment, the control processor is further configured to: i) detect a plurality of bite events based on sensor data from at least one of the capacitive touch sensor, the load cell, and the inertial measurement unit; ii) determine a bite mass value for each detected bite event; and iii) apply a decay function to an accumulated consumption signal, wherein the consumption signal at a current time step is determined using a temporally weighted integration of past bite mass values and a decay term.

In yet another embodiment, the control processor is further configured to trigger a feedback event when the accumulated consumption signal exceeds a predefined threshold.

Before undertaking the DETAILED DESCRIPTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely. Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1A is a bottom view of a smart utensil according to an embodiment of the disclosure.

FIG. 1B is a top view of a smart utensil according to an embodiment of the disclosure.

FIG. 1C is a side view of a smart utensil according to an embodiment of the disclosure.

FIG. 2A is a bottom view of a smart utensil according to an embodiment of the disclosure.

FIG. 2B is a top view of a smart utensil according to an embodiment of the disclosure.

FIG. 2C is a side view of a smart utensil according to an embodiment of the disclosure.

FIG. 3 illustrates a smart utensil in a communication system according to an embodiment of the disclosure.

FIG. 4 is a block diagram illustrating the smart utensil according to an embodiment of the disclosure.

FIG. 5 is a flow diagram illustrating a bite interval operation of the smart utensil according to an embodiment of the disclosure.

FIG. 6 is a flow diagram illustrating an image capture operation of the smart utensil according to an embodiment of the disclosure.

FIG. 7 is a flow diagram illustrating an offline usage operation of the smart utensil according to an embodiment of the disclosure.

FIG. 8 is a flow diagram illustrating a weight algorithm operation of the smart utensil according to an embodiment of the disclosure.

### DETAILED DESCRIPTION

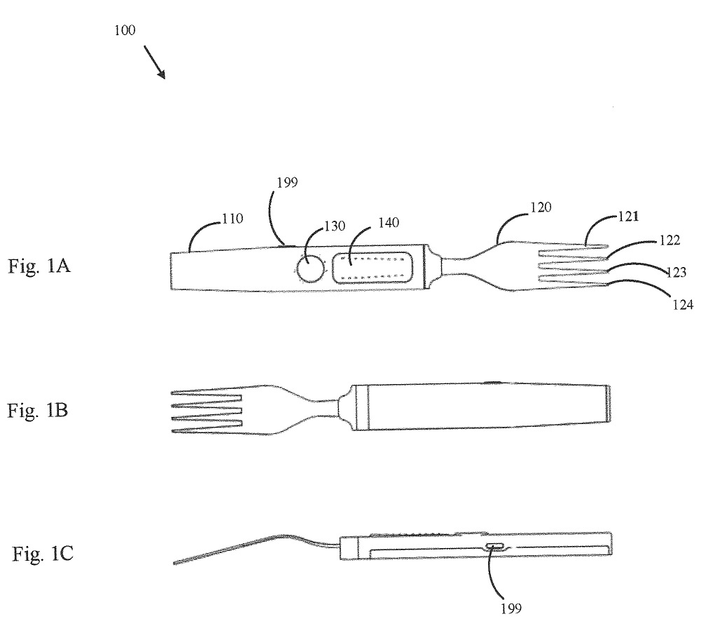

FIGS. 1 through 8, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably arranged utensil.

The present disclosure describes systems and methods using artificial intelligence (AI) to reduce the inaccuracies and inefficiencies associated with meal and nutrition tracking. The disclosed systems and methods integrate AI into an improved dining utensil that people use to eat. The disclosed smart utensil may be used as a spoon or a fork and may communicate with a companion meal-tracking application in a mobile device. The disclosed smart utensil includes various embedded sensors to determine what food is being consumed, how fast the food is being consumed, and how much is being consumed.

The disclosed system and methods provide the following capabilities: i) bite detection; ii) bite weighting, and iii) food recognition via image capture. Bite detection determined when a user places the utensil in his or her mouth using capacitive touch sensing. Bite weighing calculates the mass of food consumed by measuring the differential weight before and after a bite via a load cell. Food recognition via image capture uses an integrated camera and wireless transmission to a mobile application for AI-based food classification.

Based on this information, the disclosed system and methods compute an entire nutritional profile, including the calories, macronutrients, and micronutrients consumed during the meal. The smart utensil and the related application include technology that allows a user to track eating speed, average bite size, average time between bites, and how long the user took to complete the meals. Combining the nutritional data with user eating habits, the disclosed system and methods can do a predictive analysis to see if the user is at risk of any potential health issues in the near term.

To accomplish these objectives, the disclosed smart utensil and related application use the embedded sensors to enable a user to monitor more easily and accurately the nutritional intake of the user and to develop a more holistic view of the user's health landscape. The smart utensil includes several key components and sensors that work in conjunction to detect and accurately measure bites taken by the user during a meal cycle. The smart utensil includes a camera module, a load cell, an inertial measurement unit (IMU), a capacitive touch sensor, and a radio module (e.g., Bluetooth).

Key aspects of the smart utensil include: i) bite weight measurement, ii) food identification; iii) consumption rate feedback (bites/min); and iv) a utensil-to-app interface. Bite detection is a principal feature of the utensil that distinguishes actual user consumption from other sensor measurements. This is key to determining bite weight and providing accurate meal consumption data. The bite detection feature is implemented in an algorithm executed by a master control unit (MCU), such as a processor, that relies on multiple sensor inputs.

FIG. 1A is a bottom view of smart utensil 100 according to an embodiment of the disclosure. FIG. 1B is a top view of smart utensil 100 according to an embodiment of the disclosure. FIG. 1C is a side view of smart utensil 100 according to an embodiment of the disclosure. Smart utensil 100 comprises handle 110 and fork head 120. In the example embodiment, fork head 120 includes a plurality of tines, such as tines 121-124. Utensil 100 further includes camera 130, friction pad 140, and user control button 199.

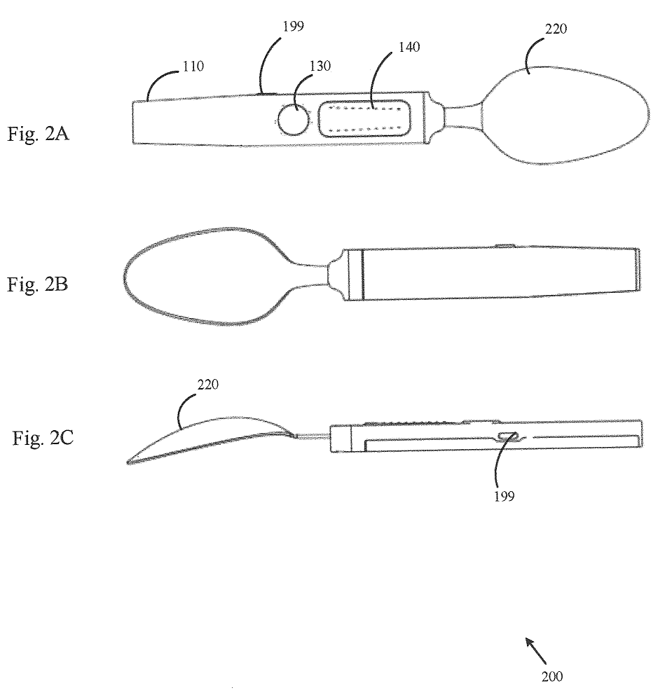

FIG. 2A is a bottom view of smart utensil 200 according to an embodiment of the disclosure. FIG. 2B is a top view of smart utensil 200 according to an embodiment of the disclosure. FIG. 2C is a side view of a smart utensil 200 according to an embodiment of the disclosure. Smart utensil 200 is similar to utensil 100 in most respects, except that the fork head 120 is replaced by spoon head 220.

The disclosed utensil 100 (or 200) includes a removable head design that improves its applicability to eating different types of food. The removable head of the device may be either a fork head 120 or spoon head 220. These metal head implementations may be coated with an electrically insulating material (e.g., anodized), except for the rear of the head, which contacts device circuitry in handle 110 and a small region on the front of the head, which contacts the mouth of the user. A capacitive touch sensing electrode on a printed circuit board (PCB) is an exposed pad to which a wire may be soldered. The other end of the wire may be made to make electrical contact with the exposed conductive region on fork head or spoon head.

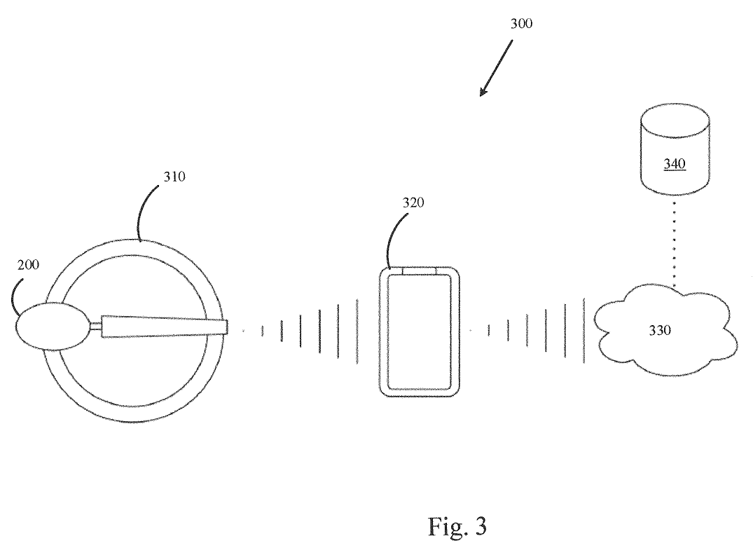

FIG. 3 illustrates smart utensil 200 (or 100) in a communication system according to an embodiment of the disclosure. In FIG. 3, utensil 200 is disposed on plate 310 and is configured to communicate wirelessly with mobile device 320 by means of, for example, a Bluetooth connection. Mobile device 320, which may be, for example, a cell phone, also communicates with image recognition server 340 via telecommunication network 330. Mobile device 320 may use a cellular connection or a WIFI connection to communicate with telecommunication network 330.

According to the principles of the present disclosure, utensil 200 may be used to capture an image of food on the dish 310 and may transfer the image to the image recognition server 340. Camera 130, such as a CMOS image sensor, is positioned near the utensil head, on the underside of the utensil. Camera 130 captures an image of the food on plate 319 before the meal begins, providing visual information about the meal. The firmware of utensil 200 transmits the captured image data to an application (e.g., via a Bluetooth connection) in mobile device and uses an AI image recognition engine in server 340 to identify the food item. A load cell on the PCB in handle 110 includes an arrangement of strain gauges integrated into the utensil head or attached to the utensil via a secure connector. When the user takes a bite, the load cell measures the bite forces and the microprocessor calculates the change in weight or force applied to the utensil head, enabling the calculation of bite weight. CMOS image sensor 130 may be alternatively positioned on the utensil, and may take images based on sensor readings or firmware state machine status as opposed to user input

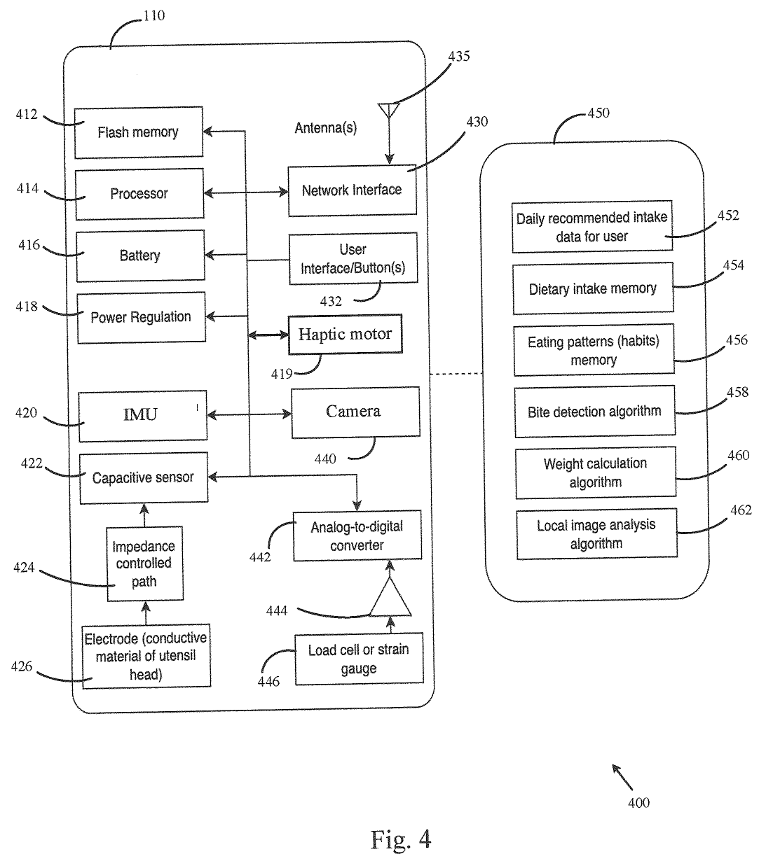

FIG. 4 is a block diagram illustrating smart utensil 400 according to an embodiment of the disclosure. Smart utensil 400 may be an implementation of a fork utensil 100 or a spoon utensil 200. The components shown in FIG. 4 may be implemented on a printed circuit (PCB) inside handle 110. Utensil 400 comprises flash memory 412, control processor 414, which functions as a microcontroller unit (MCU) 414, battery 416, power regulation 418, and haptic motor 419. Utensil 400 further includes network interface 430 (e.g., Bluetooth transceiver), antenna 435, and user interface/buttons 432, such as user button 199.

Utensil 400 also includes a package of sensors, including camera 440, which may be a CMOS image sensor 440, inertia measurement unit (IMU) 420, which comprises motion and orientation sensors, and capacitive sensor 422. By way of example, camera 440 (or CMOS image sensor 440) may be camera 130 in FIGS. 1 and 2. Utensil 400 also includes impedance-controlled path 424, electrode 426, amplifier 444, analog-to-digital converter (ADC) 442, and load cell 446.

Utensil 400 further includes firmware 450 that may be stored in flash memory 412 in handle 110. Firmware include both stored data and algorithms that are executable by control processor 414. Firmware 450 includes executable algorithm 452, which stores dietary reference intake for a user. Firmware also includes dietary intake memory 454, eating pattern (habits) memory 456, executable bite detection algorithm 458, executable weight calculation algorithm 460 and executable local image analysis algorithm 462. Bite detection algorithm 458 processes and analyzes the data from the various sensors to accurately identify and measure bites taken by the user. Algorithm 458 accounts for the interplay between the load cell readings, IMU data, and capacitive touch events to distinguish true bites from other utensil movements or disturbances.

In an exemplary embodiment, control processor 414 is a low power processor that runs continuously and performs the following functions: i) sensing by capacitive touch sensor (CTS) 422; ii) load cell operations (along with associated ADC 442); iii) inertial measurements by motion and orientation sensor 420 (e.g., 9 degrees of freedom); iv) control of haptic motor 419, and v) responding to activation of user button 199.

Control processor 414 also communicates via network interface 420, which may be a Bluetooth transceiver. Network interface 420 enables wireless communication between utensil 400 and a companion application on a mobile device, such as a smart phone. Network interface 420 and control processor 414 execute a communication protocol that handles the following functions: i) communication and pairing with the mobile application; ii) proprietary image transfer protocol; and iii) camera control and image preprocessing. Data collected by the sensors, including bite measurements and the initial food image, are transmitted to the mobile app for further analysis and feedback.

Capacitive touch sensor (CTS) 422 is configured to contact the user's mouth and trigger bite detection. Ideally, the CTS 422 may run on a dedicated circuit board with its own power regulation and isolated ground plane to minimize noise. CTS 422 uses a metallic tip of the utensil (spoon head or fork head) but may include other utensils as the capacitive electrode. CTS 422 may be tuned using pF-range discrete capacitors and may comprise a dynamically variable capacitance device to tune the subsystem dynamically during normal operation. In this way, device state latching may be prevented, and responsiveness and sensitivity may be increased.

Physical contact with the mouth alters the local electric field, increasing the capacitance along the path. This change is sensed by CTS 422 and is used to infer contact. The utensil head may be fabricated from a conductive material, as is common in conventional household utensils. This conductive substrate is advantageous for capacitive sensing applications due to its low impedance and direct interaction with the user's mouth.

To improve sensing consistency and reduce the influence of environmental parasitics, the conductive surface may be selectively covered with an insulating material. This insulating layer provides impedance control and shields non-critical regions of the conductive path, thereby minimizing unintended capacitive coupling. Strategically placed apertures or cutouts in the insulating surface, such as at the tips of fork tines 421-424 or at the apex of a spoon's convex curvature, expose localized sensing electrodes. These openings ensure reliable and consistent contact between the user's mouth and the conductive sensing regions, enhancing signal fidelity during contact detection.

In an embodiment, a touch detection electrode may be built into an integrated circuit (IC) to detect the effective capacitance of the electrode. In utensil 400, the electrode may be exposed and require an external wire linking it to the head of the device. This means the electrode (i.e., the sensing element) is subject to many stray capacitances which make ordinary tuning capacitance values of Cₓ (within the range of 2 nF to 50 nF) too large. Values on the order of tens or hundreds of picofarads (pF) are suitable. These are within the same order of magnitude as the stray capacitances of the arrangement. Because the disclosed utensil features a removable head, these stray capacitances may be a variable and dynamically adjustable tuning capacitance along with an associated tuning algorithm is used.

Load cell 446 is configured to weigh the mass of each bite by measuring the force applied to utensil head. Load cell 446 may apply some impulse response filtering to calculate mass (or weight) on the utensil 400. In an embodiment, load cell 446 may calculate a weight of each bite by capturing the pre-bite and post-bite weight values and determining the difference (e.g., delta value) between the weight values.

Load cell 446 may include an arrangement of strain gauges integrated into the utensil head or a secure mechanical connection made between the utensil (spoon or fork) head and load cell 446. A strain gauge is a resistive element whose geometry is engineered such that its resistance changes in response to axial deformation, whether tensile or compressive. Load cell 446 incorporates one or more strain gauges arranged to measure specific types of mechanical force. such as axial load, shear, or bending moment. The output of load cell 446 is a differential voltage signal, typically on the order of hundreds of microvolts. Due to the small signal amplitude, an analog amplifier (444) is required prior to digitization. This amplified signal is then passed to an analog-to-digital converter (442), enabling the measurement to be processed by a microcontroller or digital signal processor.

In an example embodiment, when force is applied downward on the head of utensil 400, load cell 446 may create a voltage differential in a Wheatstone bridge arrangement of strain gauges. This voltage differential is amplified by amplifier 444 and fed through analog-to-digital converter (ADC) 442, where it is stored for access by the master controller unit (MCU) (i.e., control processor 414) running the algorithm.

In a first embodiment, IMU 420 may comprise an integrated circuit (IC) that monitors at least five (5) degrees of freedom (DOF), including X-axis, Y-axis, Z-axis, pitch, and roll. The IMU 420 IC may be implemented on the PCB of utensil 400. Data from this IC is directly accessible to control processor 414 running the algorithm. The CTS 422 IC may also be implemented on the PCB of utensil 400. The electrode 426 or sensing element for the CTS 422 IC extends onto the head of the spoon or fork. A variable capacitive element 424 tunes the sensitivity of the CTS 422 IC.

In an embodiment, inertial measurement unit (IMU) 420 may include a 9-axis IMU (accelerometer, gyroscope) that aids in user movement classification. IMU 420 is used in conjunction with CTS 422 to progress a bite detection state machine. IMU 420 readings may also be used to compensate for the stray forces imposed on the utensil head during user movement. In an embodiment, IMU 420 includes an accelerometer and gyroscope that track the motion and orientation of utensil 400. By detecting patterns in acceleration and tilt, IMU 420 aids in distinguishing actual bites from other utensil movements (e.g., shuffling food on a plate). CTS 422 is integrated into the utensil head or a conductive element near the head. When the utensil touches the user's mouth during a bite, the capacitive sensor detects this event, helping to filter out false positives.

Camera 440 may include a downward-facing CMOS image sensor mounted on the bottom surface of the utensil 400. The user may position utensil 400 above plate 310 and press button 199 to capture an image of the food on plate 310. In an alternate embodiment, camera 440 may comprise a forward-facing camera on handle 110 for real-time image classification of individual bites.

In an embodiment, haptic motor 419 may include an eccentric rotating mass (ERM) vibration motor that provides haptic feedback to the user. The haptic feedback may be delivered under certain device conditions (e.g., battery low, failed image capture, and the like). Haptic feedback also may be provided during a meal when the user is eating too rapidly or when the meal when is approaching a daily recommended intake (DRI).

In an embodiment, utensil 400 may include a dual-microcontroller (or processor) architecture to optimize power efficiency and task distribution. A low-power microcontroller may act as the main controller, interfacing with the sensors and running the bite detection algorithm. A separate secondary microcontroller may handle peripheral functions, like camera control and wireless communication, waking up only when needed to conserve battery life.

By combining the data from the camera module (initial food image), load cell (bite weight measurements), IMU (motion tracking), and capacitive touch sensor (mouth contact detection), utensil 400 may provide users with quantitative insights into their eating habits, portion sizes, and consumption rates. This enables better monitoring and management of their nutritional intake.

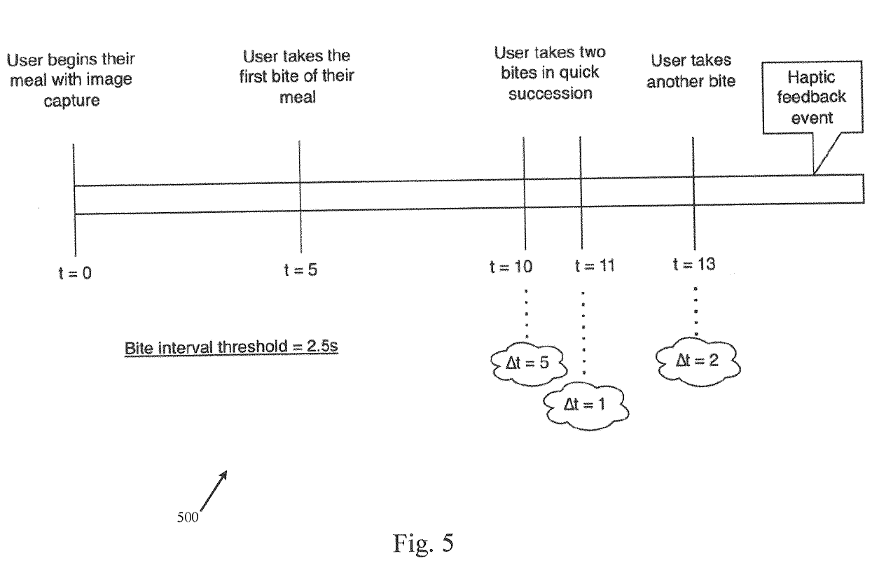

FIG. 5 is a flow diagram illustrating a bite interval operation of the smart utensil according to an embodiment of the disclosure. In FIG. 5, timeline 500 illustrates the start of a user's meal and the occurrence of the first several bites. Timeline 500 demonstrates the calculation of bite intervals and the application of a simple two-element moving average filter. A haptic feedback threshold is set at a filtered bite interval of 2.5 seconds or less. The bite interval may be defined as the elapsed time between a given bite and the preceding bite. The filtered bite interval is computed as the average of the current bite interval and the previous filtered value, according to a recursive formula:

$${y}_{n + 1} = ( {x}_{n} + {y}_{n} ) / 2 ,$$

where:
xₙ is the current measured bite interval;
yₙ is the previously filtered interval; and
yₙ₊₁ is the updated filtered value.

In an embodiment, the smart utensil may implement an additional expressive metric to enhance real-time feedback related to user eating behavior. This metric comprises a temporally weighted integration of consumed mass, wherein each bite contributes to a cumulative signal (or value) that decays continuously over time in the absence of new input events. This enables the system to reflect not only bite size but also the frequency of bites in a single dynamic signal.

This integration may be realized in discrete time by the following difference equation:

$${S}_{n + 1} = {e}^{- λΔ t} {S}_{n} + {m}_{n}$$

where:
Sₙ is the value of the consumption signal at time step n;
e^{-λΔt} is a decay term with decay constant λ, representing the exponential decay of the signal over time;
mₙ is the mass of the bite consumed at time step n.

The term mₙ is typically zero between bites, such that the integration reflects only active consumption events. Bites occurring in rapid succession cause successive contributions to accumulate constructively, resulting in higher signal values than if the same bites were spaced further apart. This produces a form of consumption momentum wherein a signal encodes both bite size and frequency.

When the integrated signal Sn exceeds a configurable threshold, a feedback event, such as haptic vibration, may be triggered to encourage moderation. In this way, the metric supports dynamic intervention strategies by estimating instantaneous bite size and short-term eating rate from recent consumption activity.

In the example in FIG. 5, the first bite occurs at 5 seconds. No interval or filtered value is available at this point.

The second bite occurs at 10 seconds, resulting in a bite interval of 5 seconds. Since no prior filtered value exists at this point, the moving average filter is initialized by assigning the filtered value to the first bite interval: x₁=y₁=5.

The third bite occurs at 11 seconds. The interval is now 1 second. The filtered value becomes: y₂=(1+5)/2=3.

Although the raw interval is below the threshold (1 sec.<2.5 sec.), the filtered value remains above the threshold (3 sec.), so no haptic feedback is triggered. The fourth bite occurs at 13 seconds. The interval is 2 seconds. The filtered value is now: y₃=(2+3)/2=2.5.

This filtered value equals the threshold, triggering a predefined haptic feedback pattern. This feedback is intended to alert the user that their eating pace may be too fast, thereby promoting mindful consumption and improved digestion.

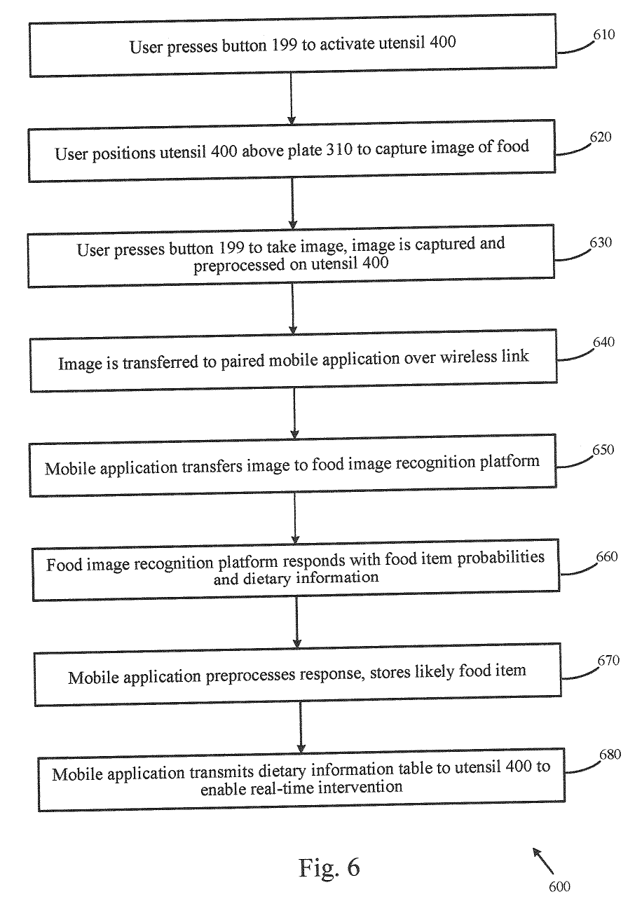

FIG. 6 is a flow diagram 600 illustrating an image capture operation of the smart utensil 400 according to an embodiment of the disclosure. In 610, a user presses button 199 to activate utensil 400. In 620, the user positions utensil 400 above plate 310 to capture an image of food. In 630, the user presses button 199 to take an image. The image is captured and preprocessed on utensil 400. Preprocessing includes black-pixel checking, possible compression techniques, and other preprocessing elements, including, but not limited to, the possibility of running inference algorithms (AI) on utensil 400 itself.

In 640, the image is transferred to a paired mobile application over the wireless link. In 650, the mobile application transfers the captured image to a food recognition platform on image recognition server 340. The mobile application uses network 330 to transfer the capture image to the food recognition platform. In 660, the food image recognition platform responds with food item probabilities and dietary information. The response may include a list of probabilities (i.e., spaghetti with meat sauce: 65%, lasagna: 23%, carbonara: 12%).

In 670, the mobile application preprocesses response, stores likely food item and retrieves nutritional data for item. The mobile application is responsible for preprocessing this list, determining if the most likely item has sufficient probability, and then retrieving nutritional data for the selected item from another or same web service(s). In 680, the mobile application transmits dietary information table to embedded device to enable real-time intervention.

Following food identification and retrieval of associated nutritional data from a remote server, macronutrient density values may be transmitted to the utensil 400 over a short-range wireless protocol (e.g., Bluetooth).

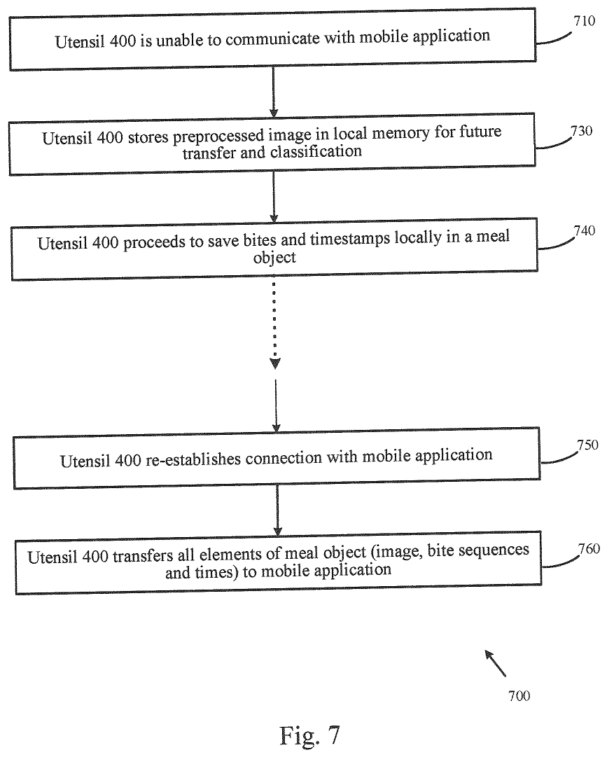

FIG. 7 is a flow diagram 700 illustrating an offline usage operation of the smart utensil according to an embodiment of the disclosure. In 710, utensil 400 is initially unable to communicate with the mobile application on the mobile phone 320. In 730, utensil 400 stores preprocessed image in local memory for future transfer and classification. In 740, utensil 400 saves bites and timestamps locally in a meal object. In 750, utensil 400 reestablishes connection with mobile application. In 760, utensil 400 transfers all elements of meal object (image, bite sequences and times) to the mobile application.

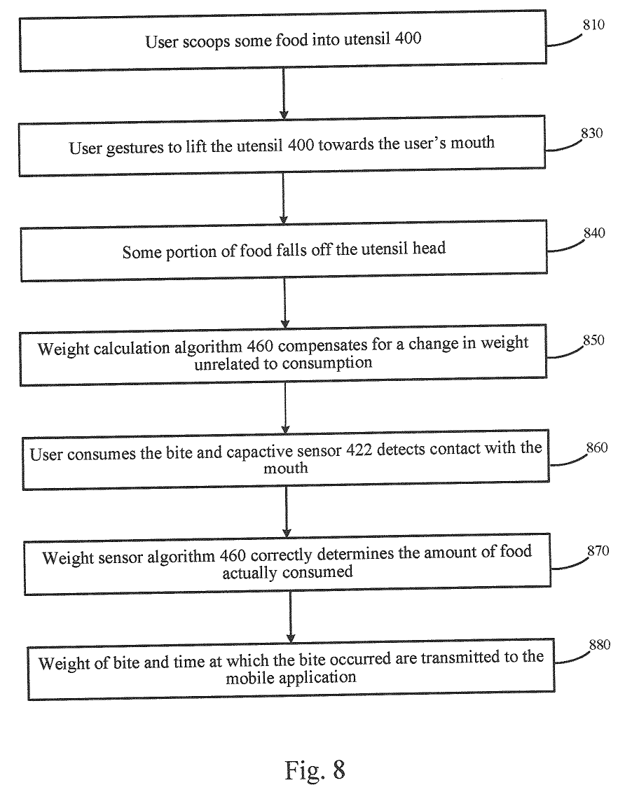

FIG. 8 is a flow diagram 800 illustrating a weight algorithm operation of the smart utensil according to an embodiment of the disclosure. In 810, a user scoops some food into utensil 400. In 830, the user lifts utensil 400 towards the user's mouth. As utensil 400 is lifted, the IMU 420 in utensil 400 detects the movement and processor 424 begins to execute weight calculation algorithm 460.

In 840, some portion of food may fall off the utensil head. In 850, the weight calculation algorithm 460 compensates for change in weight unrelated to consumption. If some food falls off the utensil head, this does not contribute to the amount of food consumed. The weight calculation algorithm 460 performs either/or some combination of an Infinite impulse response (IIR) exponential moving average filter and a simple array average. The frequency response of this filter is designed such that it is responsive enough to handle food spillage during normal consumption.

In 860, the user consumes the bite and capacitive sensor 422 detects contact with the user's mouth. In 870, the weight calculation algorithm 460 correctly determines the amount of food actually consumed. At the instant of mouth contact detection, the value registered in the load cell 446 is recorded. As the user consumes the bite, utensil 400 waits until the user has finished consuming, determined by the deactivation of capacitive touch and/or gesture detection of mouth removal. In 880, the weight of bite and time at which the bite occurred are transmitted to the mobile application.

Although the present disclosure has been described with an example embodiment, various changes and modifications may be suggested to one skilled in the art. It is intended that the present disclosure encompass such changes and modifications as fall within the scope of the appended claims.

What is claimed is:

1. A utensil for monitoring the eating habits of a user, the utensil comprising
a handle;
a head coupled to the handle and configured to receive food;
a camera module disposed in the handle and configured to capture an image of food on a plate;
a load cell coupled to the head and configured to measure force caused by food on the head;
a capacitive touch sensor configured to detect contact between the head and the mouth of a user; and
a control processor disposed in the handle, wherein the control processor is configured to determine a weight of the food on the head based on force measurements received from the load cell.

2. The utensil as set forth in claim 1, wherein the head includes a spoon head configured to be removably coupled to the handle.

3. The utensil as set forth in claim 1, wherein the head includes a fork head configured to be removably coupled to the handle.

4. The utensil as set forth in claim 1, wherein the head further comprises an inertial measurement unit disposed in the handle and configured to detect movement of the utensil.

5. The utensil as set forth in claim 4, wherein the handle further comprises a radio transceiver.

6. The utensil as set forth in claim 5, wherein the control processor is further configured to receive a captured image of food from the camera and to process the captured image using an image recognition algorithm in the handle.

7. The utensil as set forth in claim 5, wherein the control processor is further configured to receive a captured image of food from the camera and to transmit the captured image to an image recognition server via the radio transceiver.

8. The utensil as set forth in claim 7, wherein the control processor is further configured to determine a time period between user bites based on signals received from the capacitive touch sensor.

9. The utensil as set forth in claim 8, wherein the control processor is further configured to:
detect a plurality of bite events based on sensor data from at least one of the capacitive touch sensor, the load cell, and the inertial measurement unit;
determine a bite mass value for each detected bite event; and
apply a decay function to an accumulated consumption signal, wherein the consumption signal at a current time step is determined using a temporally weighted integration of past bite mass values and a decay term.

10. The utensil as set forth in claim 9, wherein the control processor is further configured to:
trigger a feedback event when the accumulated consumption signal exceeds a predefined threshold.

11. A utensil for monitoring eating behavior of a user, the utensil comprising
a plurality of sensors configured to detect multiple bite events;
a control processor configured to:
determine a bite mass value for each detected bite event;
apply a decay function to an accumulated consumption signal, wherein the consumption signal at a current time step is determined using a temporally weighted integration of past bite mass values and a decay term; and
trigger a feedback event when the accumulated consumption signal exceeds a predefined threshold.

12. The utensil as set forth in claim 11, wherein the utensil includes a capacitive touch sensor configured to detect contact between the utensil and the mouth of a user.

13. The utensil as set forth in claim 12, wherein the utensil includes a load cell configured to measure force caused by food on the utensil.

14. The utensil as set forth in claim 13, wherein the utensil includes an inertial measurement unit configured to detect movement of the utensil.

15. The utensil as set forth in claim 11, wherein the decay function is given by the difference equation:
$${S}_{n + 1} = {e}^{- λΔ t} {S}_{n} + {m}_{n}$$
where:
Sₙ is the value of the consumption signal at time step n;
e^{-λΔt} is a decay term with decay constant λ, representing the exponential decay of the signal over time;
mₙ is the mass of the bite consumed at time step n.
